(19) United States
(12) Patent Application Publication
(10) Pub. No.: US 2013/0058884 A1
(43) Pub. Date: Mar. 7, 2013

(54) HERBAL OIL WHICH IS USED IN HAIR CARE AND IN MASSAGE AND WHICH IS USED IN TREATMENT OF HAIR LOSS, HAIRLESSNESS, AND BURNS

(71) Applicant: Münevver Yalcin, Adana (TR)

(72) Inventor: Münevver Yalcin, Adana (TR)

(21) Appl. No.: 13/579,146

(22) PCT Filed: Dec. 13, 2010
(86) PCT No.: PCT/TR2010/000244

§ 371 Date: Oct. 1, 2012

(30) Foreign Application Priority Data
Feb. 18, 2010 (TR) 2010/01251

(51) Int. Cl. A61K 8/97; A61Q 5/00; A61P 25/00; A61Q 19/00; A61P 17/02; A61K 36/752; A61Q 7/00

(52) U.S. Cl. 424/74; 424/736

(57) ABSTRACT

A compound which is obtained by mixing olive oil with natural vegetables and their essentials which are used in hair care and which prevents hair loss, removes deformation and allows healthy hairs to regrow if hair has been lost, allows new hairs coming out when it is applied where hairlessness has occurred, and which, also allow pains from sunburn, skin burn, and fire burn stopping and removing completely sears from such burns. The compound may also be used as massage oil.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The discovery is related to the compound which is obtained by mixing olive oil with natural vegetables and their essentials which are used in hair care and which prevent hair loss, remove deformation and allow regrowth of healthy hairs if hair has been, lost, allow new hairs coming out when it is applied where hairlessness has occurred, and which also allow pains from sunburn, skin burn, and fire burn stopping and allow sears from such burns removing completely as well as using as massage oil.

2. Description of Related Art Including Information Disclosed Under 27 CFR 1.97 and 37 CFR 1.98

Having healthy hairs and protecting healthy hairs is significant, and not achieving this and the hair loss and hairlessness for any reason whatsoever affects human psychology negatively and as a consequence it affects life quality negatively. There are numerous works and products in order to prevent hair loss and/or to is grow lost hairs. In this subject, harmful dermatological chemical mixtures exist in the market and in the developed technologies and some nuisance processes for peoples. Hair transplantation that is quite expensive and whose results cannot be doped, the chemical compounds consisting of various chemicals, and some vegetables are used together with some mixtures containing chemical agents.

Furthermore, burns and sears after burns are also serious health problem for individuals. Preventing sunburns stopping pains from sunburns, liquid burns, and fire burns, and removing any sears from those on the skin are important health problems. For purpose of removing these problems, pharmaceutical firms use various chemicals. Compounds used in this issue contain chemical agents and cause negative results for human health.

### BRIEF SUMMARY OF THE INVENTION

The present invention is made with complete natural methods by combining the products, which are completely obtained, from the nature. Therefore it is different from present products which contain chemical admixtures and it does not arise any risk for health and also gives very effective results in relevant areas.

Moreover, because the present invention softens the skin it prevents chaps which may occur during massage and allows healthier rubbing by opening pores.

The formula of the complete vegetable and nature oil which has been developed for accomplishing foregoing results is following:
Blackberry leaf and scape (3000 g)
Lemon leaf and scape (500 g)
Bitter orange leaf and scape (1250 g)
Mandarin leaf and scape (1000 g)
Bermuda grass stolon root (1250 g)
Grape seed (100 g)
Purslane and its root (8000 g)
Mallow seed (350 g)
Bay leaf and root (1500 g)
Pomegranate leaf and scape (2500 g)
Agropyron repens (1250 g)
Basil leaf and scape (500 g)
Jasminum sambac (25 g)
Lemon blossom (125 g)
Orange blossom (125 g)
Onosis and its root (1250 g)
Couscous and its root (2500 g)
Fresh fruit of lemon (10 pieces)
Blackberry blossom and sptrout (3000 g)
Daisy leaves scape and root (200 g)
Stinger (1000 g)
Olive oil (40.000 g)

### DETAILED DESCRIPTION OF THE INVENTION

The compound of the present invention has been made by combining the products whose names and rates are written above and which are completely natural is started by preparing the olive oil.

Preparing the product:

First, green olives are gathered front one garden for the olive oil which will be used and the minerals therein are ensured not died using cool pressing technique in the same day and their oil are taken off. Obtained oils are rested 1 day in a dark color, glass jar in cool and shadowed place at height.

A large jar is used for forming the compound of the present invention. Placing said vegetables in that jar in the indicated order is very important because these vegetables may deform such as mossiness and bruising.

Vegetables which are reaped from the natural environment by hand one by one are washed with clean and flushing water. They are rested until their juice is dried without draining their juice not exposing to extreme sunlight.

Blackberry scapes are sliced up 5-6 cm pieces and they are put in the bottom of an empty and clean jar together with their leaves. Lemon leaves and its scapes are sliced in the same way and put in the jar. Bitter orange leaves and scapes and mandarin leaves and scapes are sliced in the same way and put in the jar. Then, Bermuda grass stolon root is put on foregoing items. Seed of fresh grapes that have been dried are crumbled in a wooden mortar and are pulverized and they are sprinkled above indicated items. Purslanes are split together with their roots which have at least 1 cm diameter and put on the other vegetables in the jar. Small black seeds of purslanes which have remained on the water while they were washed are added to mixture together with the purslanes and their roots. Seeds and leaves of mallow and its scapes having at least 2 cm diameter are placed and then one half of bay leaves (750 g) which is used in the mixture are added to them. Leaves of the pomegranates and their scapes having at least 5 cm diameters are put on above indicated items. The agropyron repens are put on them. Basil leaves and seeds are split and put on above mentioned items. Later, jasminum sambac, lemon blossoms, orange blossoms, onisis and its roots, couscous and its roots, blackberry blossoms and seeds, daisy leaves and scrapes (their blossoms are not used), stingers, and the remained bay leaves are put, and fresh fruits of lemon (10 pieces in size of walnut) are put on them. The process is completed. Olive oil is added to these vegetables, their roots, and fruits and so the vegetables are prevented from contacting air.

The glass jar that has been tightly closed is checked in a cool and shadowed place every day and is left to rest for 4 months. One week after the application, the jar is opened and lemon juices are removed from the mixture and then it is closed again. The vegetables are ensured not lifting on the oil and not becoming suberized. At the end of the 4th month the vegetables are filtered from the oil and that oil is left to rest. Seeds of grapes and purslane seeds are kept in the oil and left to rest for one more month and then are filtered by thin sieve and removed from their feculas. Rose oil or lemon blossom oil is put in the remainder mixture for obtaining pleasant odour and then they are put in little bottles and are made ready for sale.

The product is applied as following:

While using it in burns, applying the product on the burnt area 5 times per day removes the pain from the burn and also closes the sears. Furthermore the product relating to the discovery can be used as preventive against sunburns.

While using it in massage oil, applying the product prior to massage on the area which will be rubbed allows the pores opening and it also feed the skin together with the massage.

As the main usage area of the product, in order to prevent hair loss and to ensure new hairs to come out if hairlessness has occurred, it is applied on hairs and skin once a day. The product that is applied everyday is waited 2-7 hours. For obtaining optimum benefit, the waiting period should be maximum.

For cleaning, hairs are fairly good washed with olive oil essential soap or shampoo.

1. A vegetable oil which is used in hair care and which prevents hair loss, removes deformation and allows healthy hairs re-coming out if hair has been lost, allows new hairs coming out when it is applied where hairlessness has occurred, and which also allows pains from sunburn, skin burn, and fire burn stopping and sears from such burns removing completely and as well as using as massage oil, the oil containing the following items:
Blackberry leaf and scape (3000 g);
Lemon leaf and scape (500 g);
Bitter orange leaf and scape (1250 g);
Mandarin leaf and scape (1000 g)
Bermuda grass stolon root (1250 g);
Grape seed (1100 g);
Purslane and its root (8000 g);
Mallow seed (350 g);
Bay leaf and root (1500 g);
Pomegranate leaf and scape (2500 g);
Agropyron repens (1250 g);
Basil leaf and scape (500 g);
Jasminum sambac (25 g);
Lemon blossom (125 g);
Orange blossom (125 g);
Onosis and its root (1250 g);
Couscous and its root (2500 g);
Fresh fruit of lemon (10 pieces);
Blackberry blossom and sprout (1000 g);
Daisy leaves scape and root (200 g);
Stinger (1000 g); and
Olive oil (40.000 g).

2. A production method of the vegetable oil of claim 1, comprising the following steps:
First, green olives are gathered from one garden for the olive oil which will be used and the minerals therein are ensured not died using cool pressing technique in the same day and their oil are taken off; obtained oils are waited 1 day in a dark color glass jar in cool and shadow place at height; a large jar is used for forming the compound of discover; placing said vegetables in that jar in indicated order is very important because these vegetables may deform such as mossiness and bruising; vegetables which are reaped from the natural environment by hand one by one are washed with clean and flushing water; they are waited until their juice is dried without draining their juice not exposing extreme sunlight; blackberry scapes are sliced up 5-6 cm pieces and they are put in the bottom of an empty and clean jar together with their leaves; lemon leaves and its scapes are sliced in the same way and put in the jar; bitter orange leaves and scapes and mandarin leaves—and scapes are sliced in the same way and put in the jar; then, bermuda grass stolon root is put on foregoing items; seed of fresh grapes that have been dried are crumbled in a wooden mortar and are pulverised and they are sprinkled above indicated items; purslanes are split together with their roots which has at least 1 cm diameter and put on the other vegetables in the jar; small black seeds of purslanes which have remained on the water while they were washed, are added to mixture together with the purslanes and their roots; seeds and leaves of mallow and its scapes having at least 2 cm diameter are place and then one half of bay leaves (750 g) which is used in the mixture are added to them; leaves of the pomegranates and their scapes having at least 5 cm diameters are put on above indicated items; the agropyron repens are put on them; basil leaves and seeds are split and put on above mentioned items; later, jasminum sambac, lemon blossoms, orange blossoms, onisis and its roots, couscous and its roots, blackberry blossoms and seeds, daisy leaves and scrapes (their blossoms are not used), stingers, and the remained bay leaves are put and fresh fruits of lemon (10 pieces in size of walnut) are put on them; olive oil is added to these vegetables, their roots and fruits which have been put in above order; the glass jar that has been tightly closed is checked in a cool and shadow place every day and is left to rest for 4 months; one week after the application, the jar is opened and lemon juices are removed from the mixture and then it is closed again; the vegetables are ensured not lifting on the oil and not becoming suberized; at the end of the 4th month the vegetables are filtered from the oil and that oil is left to rest; seeds of grapes and purslane seeds are kept in the oil and left to rest for one month and then are filtered by thin sieve and removed from their feculas; rose oil or lemon blossom oil are put in the remainder mixture for obtaining pleasant odour and then they are put in little bottles and are made ready for sale.
